(19) United States
(12) Patent Application Publication
(10) Pub. No.: US 2007/0160515 A1
(43) Pub. Date: Jul. 12, 2007

(54) Manually activated penetrating hand sterilizer

(76) Inventor: John Harry Mohrman, Orwigsburg, PA (US)

Correspondence Address: John Harry Mohrman, 301 Village Road, Orwigsburg, PA, 17961, US

(21) Appl. No.: 11/405,127

(22) Filed: Apr. 17, 2006

Related U.S. Application Data
(60) Provisional application No. 60/750,623, filed on Jan. 9, 2006.

(51) Int. Cl. A61L 2/18

(52) U.S. Cl. 422/300

(57) ABSTRACT

A manually activated penetrating hand sterilizer has been developed as preferred state-of-the-art to improve the means of minimizing or eliminating the bacteria count on the human hands. This improved method sterilizes the primary portion of the hands, skin creases, the top portion the fingernails and under the fingernails. An antibacterial liquid is used under pressure by air compression means to form a liquid mist that is circulated in and around the hands to assure such penetration. When the liquid mist ends the circulating air continues until the hands are dry. Inserting the hands into the sterilizing compartments through the slit in the woven flexible material covering the front of those compartments, thereby pulling an embedded cord that turns a spring-tensioned switch on starting an electric motor and air compressors starts the sterilizer. The antibacterial fluid is forced out of a now pressurized tank to form a mist through a multiplicity of jets. The mist is circulated within the compartments around and penetrating the hands by air through the directional ducts emanating from the air compressors. The sterilizer operation stops when the hands are dry and are withdrawn from the compartments.

### CROSS-REFERENCE TO RELATED APPLICATION

U.S. Pat. No. 6,141,811 Nakamura

Ser. No. 60/750,623 * January 2006 * Mohrman

### STATEMENT REGARDING FEDERALLY SPONSORED RESEARCH OR DEVELOPMENT

Not Applicable

### REFERENCE TO MICROFICHE APPENDIX

Not Applicable

### BACKGROUND OF THE INVENTION

This invention relates to a manually activated penetrating hand sterilizer that minimizes or eliminates the bacteria count in skin creases, the top portion of fingernails, under the fingernails and the primary portion of the hands, thereby assuring that the subject's hands are sterilized and meet or exceed the requirements of any Governmental Health Agency. The atomized mist spray of an antibacterial fluid permeates the entire surface of the hands when placed in the sterilizing compartments of the invention thereby assuring that maximum sterilization is attained.

Past efforts to develop methods of hand sterilization have proven to be only partial at best because coverage of the entire hands was not accomplished. In attempting to remove dirt and bacteria from the hands in the past only methods of either scrubbing with antibacterial soap and regular water, using hazardous light rays with sprays that did not assure complete coverage or wiping a person's hands with antibacterial wipes were available. Most past methods used to sterilize a person's hands were time-consuming and inefficient and could not satisfactorily eliminate the bacteria to an acceptable level from difficult areas of skin wrinkles and around fingernails and other parts of the hands. Maintaining maximum bacteria free hands for persons in households, medical care facilities and other public places has been a problem due to the constant contact with the multitude of items that are subject to being touched by the hands. Hence, the hands are the primary method of contact with other germ-laden objects or persons and the carrier of such germs. Further, all methods employed to date to sterilize hands to the degree that maximum bacteria have been removed are unsuccessful. Immediately after using some of the existing methods of partial hand sterilization the hands again become contaminated in touching almost any object.

### SUMMARY OF THE INVENTION

The purpose of the invention is to provide a means to allow a person to sterilize their hands thereby reducing the bacteria count thereon to a minimum. Thus, by having an antibacterial mist penetrate each crevice and crack of the front and back of the fingers, fingernails and other parts of the hands when hands are not soiled, maximum sterilization of the hands is accomplished. The present invention comprises a mechanism that is extremely efficient and simple to operate.

In a preferred embodiment of invention, there is provided a wall-mounted assembly with two front areas covered with a flexible woven material impervious to moisture. Each area has a larger upper section with a smaller lower section. The upper section overlaps the lower section inside the sterilizing compartments. The bottom of each upper section contains a seam to house a starting cord that is anchored at the center section of the compartments and protrudes through grommets in the far left and far right outer compartment walls to spring-tensioned starting switches. Both the left and the right inner compartments contain upper and lower grids to restrict the hands from touching the multiplicity of mist jets on the top and bottom walls of the sterilizing compartments. Directional air vents are installed in the right and left outer walls of the inner sterilizing compartments. In operation, when hands are inserted into the sterilizing compartments through the openings between the front right and left upper and lower flexible woven material, the starting cords are arched over their hands and wrists turning on spring tensioned switches to an electric motor driving the air compressors located behind the sterilizing compartments within the housing assembly. The air compressors pressurize a small tank forcing the antibacterial liquid through the jets while simultaneously the air compressors force air through directional air ducts to both compartments causing the antibacterial mist to swirl in and around the hands. In this process the air pressure entering the tank forces an inlet valve in the supply line from a reservoir tank to close and an outlet valve in the supply line to the multiplicity of mist jets to open. When the tank containing the antibacterial liquid is emptied the air compressors continue to operate until the hands feel dry and can be withdrawn from the compartments thereby allowing the starting cord to return to a horizontal position and the spring-tensioned switches are turned off causing the electric motor and air compressors to stop operating. Concurrently, the spring operated inlet valve for the liquid reservoir opens and the spring operated outlet valve in the supply line to the multiplicity of mist jets close. When the invention is in use, the air compressor air intakes located at the bottom rear of the housing assembly allow filtered air to enter for use as described. An exhaust assembly tray containing liquid absorbing material is mounted on the bottom of the housing assembly with exhaust openings in the bottom of each inner sterilizing compartment. The large antibacterial liquid reservoir container inside the top of the housing assembly is refilled by removing a key-lock cap located on the top rear of the housing assembly. An electrical switch to control power to the housing assembly is located on the right side. The top, front and bottom of the housing assembly form one section as a cover and are hinged at points adjacent to the wall on top to allow access for maintenance purposes. However, for security, the key-lock cap located on top of the assembly must be removed in order for the assembly cover to be raised up.

### BRIEF DESCRIPTION OF THE SEVERAL VIEWS OF THE DRAWING

FIG. 1 is a schematic of a front view of the complete housing assembly with wall mounts and outlines of the hand sterilizing compartments.

FIG. 2 is a side sectional view of the components contained in the housing assembly.

FIG. 3 is a broken out, more detailed rear view of all of the components in the housing assembly taken along lines 3-3 of FIG. 2.

FIG. 4 is a broken out, more detailed front view of all of the components in the housing assembly taken along lines 4-4 of FIG. 2.

FIG. 5 is a broken out, more detailed bottom view of all of the components in the housing assembly taken along lines 5-5 of FIG.2.

FIG. 6 is an enlarged schematic illustrating detail of the spring operated control valves for liquid supply.

FIG. 7 is an enlarged schematic illustrating detail of liquid spray jets and grid components in the sterilizing compartments.

### DETAILED DESCRIPTION OF THE INVENTION

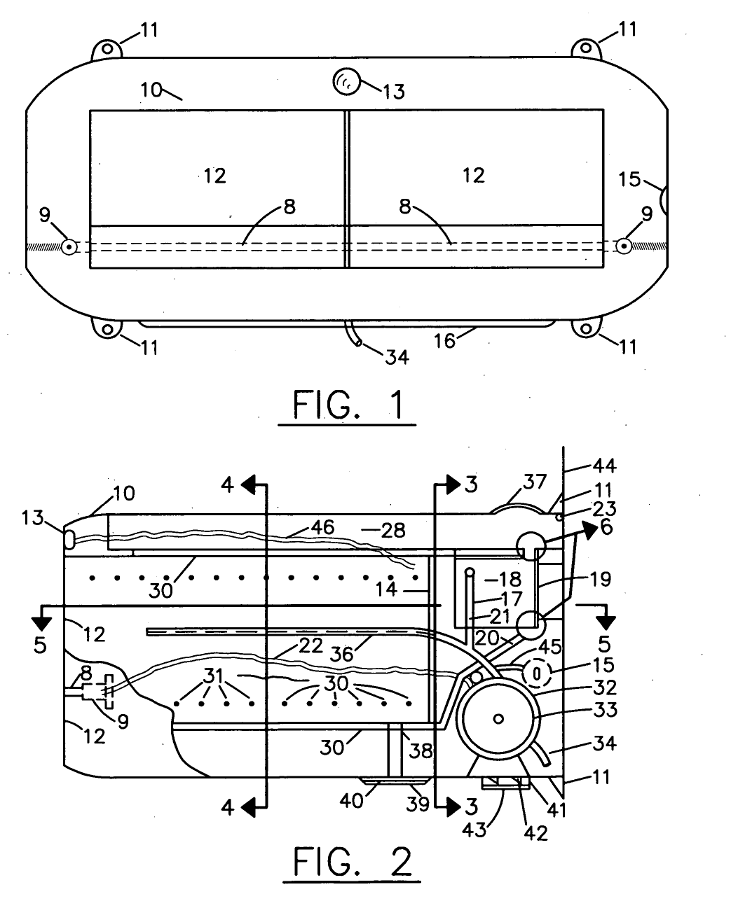

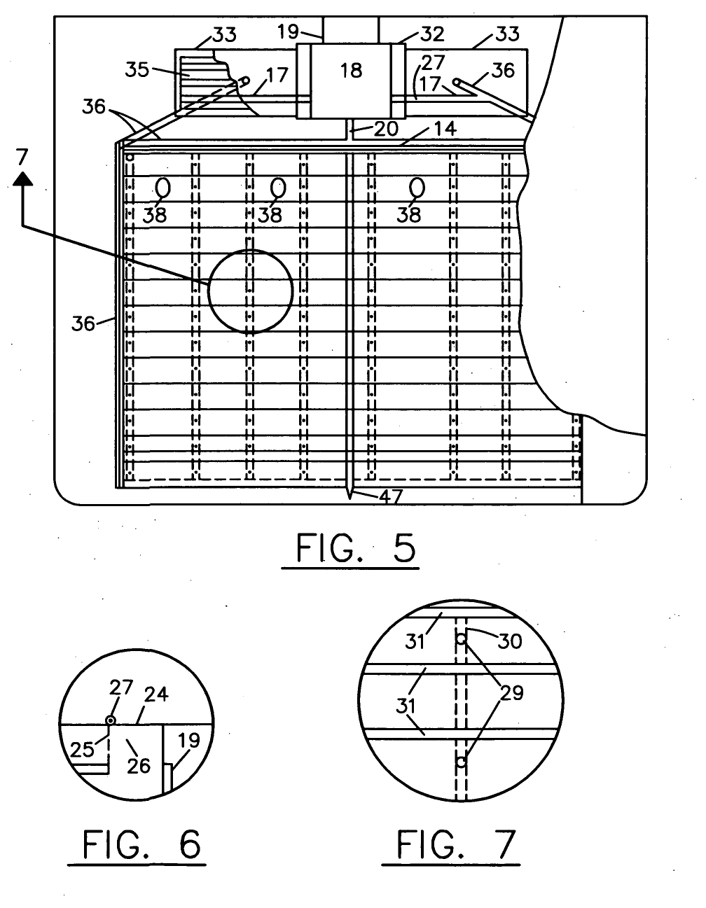

The functional components of the manual activated penetrating hand sterilizer are depicted in FIGS. 1-7. In the view in FIG. 1 of the housing assembly, 8, the phantom starting cords, 9, are the spring tensioned switches, 10, is the housing assembly, 11, is the wall brackets, 12, are the flexible woven upper and lower sections of material impervious to moisture that allows the hands to be inserted through the split opening, 13, the light indicating the unit is in operation, 15, is a phantom view of the electric control switch and 16, is the partition between compartments, 39, is the air exhaust assembly tray containing liquid absorbing material. FIG. 2 which is a side sectional view again showing 10, as the housing assembly, 11, as the wall brackets, 12, the flexible woven upper and lower sections of material impervious to moisture, 13, the light, when on, indicating the unit is in operation, 14, the sterilizing compartments with the rear sections covered by a resilient material to protect fingernails, 15, is a phantom view of a primary electric control switch, 16, is the partition between compartments, 39, is the air exhaust assembly tray containing liquid absorbing material, 17, is the air pressure line from compressors to force the antibacterial liquid from the small tank 18, 19, is a support bracket for the small tank fastened to the rear section on the housing assembly 10, 20, is a directional valve in the antibacterial liquid supply line 30, 21, is a direction valve in the air supply line to tank 18, 22, is wiring from 9, the spring tensioned starter switches to the electric motor 32, 23, is the upper hinge to allow the outer cover of the housing assembly 10 to swing upward for servicing, 28, is the antibacterial reservoir tank, 30, are the pipelines carrying the antibacterial fluid to the sterilizing compartment mist jets 29, (FIG. 7), 31, are end views of the multiplicity of grid members, (FIG. 6), 32, is the electric motor that drives compressors 33, and said motor obtains electric power through cord 34, 36, are the compressor air vents to the sterilizing compartments 14, 37, is the key-lock cap to access the antibacterial liquid reservoir 28, 38, are the air exhaust vent openings in the bottom of the sterilizing compartments 14, 39 is the exhaust assembly tray containing liquid absorbing material 40, 41, is the air compressor air intake enclosure to allow air to be drawn through the filters, 42, are the air intake openings, 43, are the filters for the air intake enclosure, 44, shows the wall to which the housing assembly would be attached, 45, is wiring from switch 15 to electric motor 32, 46, is a wiring connection between the light 13 and electric motor 32.

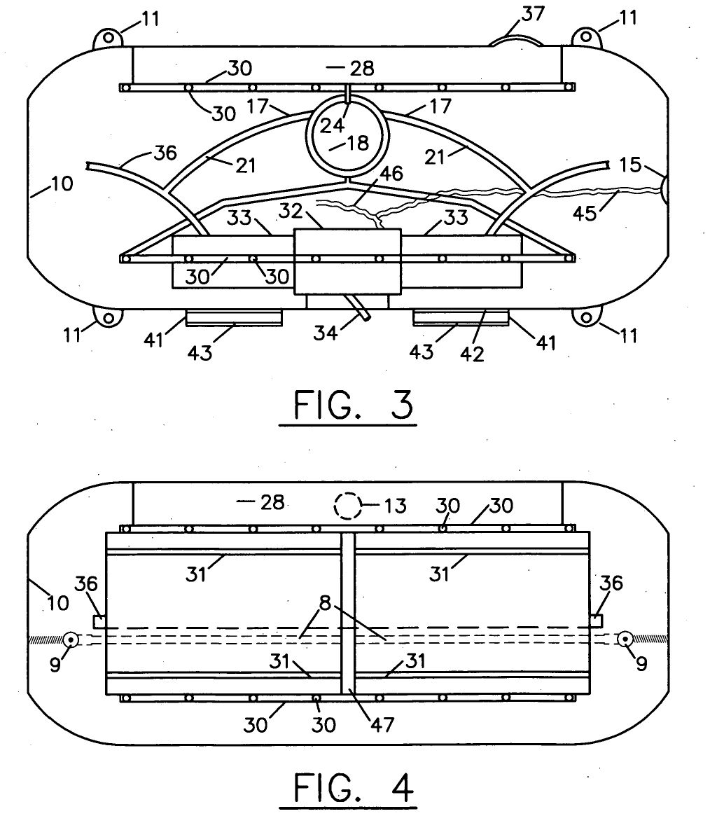

FIG. 3, taken along lines 3-3 of the FIG. 2, shows the mounting brackets 11, for the housing assembly 10, the electrical switch 15, the inner distribution lines from air compressors, 33, as, 17, with directional valves in said lines as 21, the inlet and outlet liquid control valves 24 in tank 18, the electric motor 32, the electric power cord 34, air distribution lines 36, to supply air through directional ducts to the sterilizing compartments 14, the electric motor as 32, 41 is the air compressor enclosure, 42, in the air intake openings, 43, are the filters for the air intake, 45, and 46, represent wiring to the light in the housing assembly to the primary switch controlling electricity to the unit.

FIG. 4, taken along lines 4-4 of the FIG. 2, shows the starting cord, 8, connected to the spring tensioned switches 9, the housing assembly as 10, a phantom representation of a light 13, the antibacterial reservoir supply tank 28, manifold supply lines for the

I claim:

1. In a hand sterilizing system that comprises insertion of hands into front openings thru to the rear portion of sterilizing compartments thereby activating an electrical switch causing air compressors to operate by air pressure forcing antibacterial liquid from a pressure tank into sterilizing compartments while simultaneously causing pressurized room temperature air to enter the sterilizing compartments causing the hands to dry as the antibacterial liquid stops flowing from the pressurized tank and hands withdrawn from the sterilizing compartments are dry and sterilized, the improvement comprising:
(a) The front of the sterilizing compartments are equipped with a flexible woven moisture-resistant material that contains two parts each seamed at the opening with the bottom section containing a stationary elongated cord to form a firm horizontal edge while the upper seam is equipped with a cord that is attached to a spring on one end and a spring-tensioned electrical switch means on the opposite end;
(b) An antibacterial liquid reservoir supply tank provides for a multiplicity of uses of the sterilizer by refilling the smaller pressure tank as it empties with the inlet valve means open to allow antibacterial liquid to flow from the reservoirs supply tank while the outlet valve means from the pressure tank to the sterilizing compartments is closed;
(c) The sterilizing compartments contain multiple horizontal aligned rods at the bottom and top equally spaced as hand rests and identical horizontally aligned rods equally spaced above with spaced grids above and below those rods contain numerous jet openings that serve to form liquid mist along with a resilient material at the back of the compartments to protect finger ends;
(d) The inside of the outer walls and both sides of the center wall of the sterilizing compartments contained compressed directional air openings to cause the antibacterial liquid mist to move in a swirling motion for maximum penetration of cracks and crevices of hands and as the pressurized tank becomes devoid of liquid the forced air continues to flow until the hands dry;
(e) The operating compressors provide air pressure via piping to the pressure tank forcing valve means to activate thereby forcing a selective quantity of antibacterial liquid out of the tank through distribution conduit to the jet mist openings while simultaneously distributing compressed air into the sterilizing compartments;
(f) As dry hands are withdrawn from the sterilizing compartments the spring tensioned switch returns to the off position terminating operation of compressors thereby allowing valve means at the inlet and outlet of liquid pressure tank to operate to allow the pressure tank to be filled from the reservoir supply tank for subsequent use of the sterilizer;
(g) Each sterilizing compartment contains openings at the rear bottom of the housing to provide air exhausts equipped with a receptacle on the outer bottom of the unit containing replaceable absorbent material to collect excessive air and moisture that may emanate from the compartments.

2. The bottom of the sterilizing compartments is also the base section for mounting ancillary functional equipment and the base for the housing that covers the sterilizing compartments, the improvement comprising:
(a) Openings in the back of the base allow filtered air to enter the housing for the compressor air intake and is equipped with an appropriate air filter enclosed in an accessible compartment to allow filter replacement under the bottom;
(b) The housing consists of two sides, a front section that contains openings to allow the hands to be inserted in the sterilizing compartments and is attached to the top section that is hinged to a back section that is wall mounted and is primary support for the base of the housing;
(c) Access to the antibacterial liquid reservoir tank is through an opening on top of the housing and when closed a lock type cap that attaches to the top of the sterilizing compartment to prevent access to the housing interior;
(d) The lock type cap removal permits the front and top sections to swing upward toward the mounting wall as necessary to access the ancillary components of the unit;
(e) The front housing section contains a light bulb that lights up when the unit is in operation and is off as the unit stops operating.

3. Use of the sterilizer allows hands to remain in a resting position while being treated.

4. The versatility of the sterilizer operated in appropriate locations will have a positive effect on reducing hand-borne bacteria.

5. The variable elapsed time of using the sterilizer in operation depends on the designed capacity of the liquid pressure tank.
